(19) United States
(12) Patent Application Publication
(10) Pub. No.: US 2010/0286574 A1
(43) Pub. Date: Nov. 11, 2010

(54) TREATING APPARATUS

(75) Inventors: Brian Walsh, Alpharetta, GA (US); Ralph Senft, Bavaria (DE)

Correspondence Address: KING & SPALDING, 1180 PEACHTREE STREET , NE, ATLANTA, GA, 30309-3521, US

(73) Assignee: DORNIER MEDTECH SYSTEMS GMBH, Wessling (DE)

(21) Appl. No.: 12/161,090

(22) PCT Filed: Jan. 16, 2007
(86) PCT No.: PCT/EP2007/000343

§ 371 Date: Mar. 24, 2010

(30) Foreign Application Priority Data
Jan. 17, 2006 (DE) 10 2006 002 273.4

(51) Int. Cl. A61B 17/22

(52) U.S. Cl. 601/4

(57) ABSTRACT

The present invention relates to a treating apparatus which can be used with a radiation source and which can particularly be employed in lithotripsy, a shockwave source being provided which generates shockwaves and with which shockwaves can be sent to an object to be treated, and a radiation locating device being also provided which is integrated into the shockwave source. To provide a treating apparatus of this type which despite the use with a radiation source permits a high image quality and accurate focusing of the shockwaves in a way which is as easy as possible, the radiation locating device comprises a radiation receiving unit which is configured as a solid-state detector. The solid-state detector can include both radiation locating units and ultrasonic locating units.

### RELATED APPLICATIONS

This non-provisional patent application claims priority to PCT International Patent Application No. PCT/EP2007/000343, entitled “Treating Apparatus,” filed Jan. 16, 2007, which claims priority to German Patent Application No. DE 10 2006 002 273.4 filed Jan. 17, 2006. The complete disclosure of each of the above-identified priority applications is fully incorporated herein by reference.

### TECHNICAL FIELD

The present invention relates to a treating apparatus which can be used with a radiation source and can particularly be employed in lithotripsy.

### BACKGROUND

A generic treating apparatus is known from U.S. Pat. No. 5,795,311. The treating apparatus comprises a shockwave source as a wave source which has an X-ray locating device integrated thereinto. The X-ray central beam thereby extends through the treating apparatus.

It is known from DE 39 16 093 A1 how such a treating apparatus is arranged in an overall system. The X-rays emitted by the X-ray locating device of the shockwave source are received by an image intensifier located opposite to the shockwave source and are processed into a visible image. In the shockwave source the X-ray radiation must pass through the coupling liquid contained in a coupling bellows. However, this process attenuates the X-ray radiation, which reduces the image quality.

As a remedy, it is suggested that an air-filled tube should be used. Such a tube displaces the coupling liquid in the area of the emitted X-ray beam cone, thereby improving the image quality. The tube, however, impairs the focusing of the shockwaves produced by the shockwave source, for it displaces not only the coupling liquid needed for propagating the shockwaves, but additionally creates objectionable reflection areas that scatter the shockwaves, i.e., counteract the focusing thereof.

Accordingly, a need exists in the art to provide a generic treating apparatus which can be used with a radiation source and provides for a high image quality and a high output of the pressure waves in a way that is as simple as possible.

### SUMMARY

According to the invention said object is achieved with a treating apparatus comprising the features of claim 1.

Thanks to the integration of the radiation receiving unit in the wave source, the image recording plane is positioned close to the object to be treated with the wave source. Such closeness augments image definition. Further, the radiation receiving unit can be configured in a compact way as a solid-state detector. It is thereby possible to substantially maintain the dimensions of the wave source despite the integration. The solid-state detector permits the use of small radiation doses and opens up possibilities for computerized image processing.

Advantageously, the solid-state detector can be arranged behind a wave generator of the wave source with respect to the main pressure-wave emission direction. As a result, the solid-state detector can be made relatively large in the wave source, whereby a large radiation entry area can be covered. Even if parts of the wave generator are concurrently imaged, a sharp image is nevertheless achieved.

Preferably, the solid-state detector can be arranged with respect to the main pressure-wave emission direction of the wave source in front of a wave generator of the wave source. The image remains free from shadowing by the wave generator. Surprisingly enough, a sufficiently large radiation entry area can also be sensed with this arrangement with a high image quality.

Preferably, the solid-state detector may be arranged to be integrated into a wave generator of the wave source. The pressure waves can thus be emitted unaffected by the solid-state detector. Despite the constructional adaptation of the solid-state detector with respect to the wave generator, it is possible to sense a sufficiently large radiation entry area with a high image quality.

Advantageously, the solid-state detector can be arranged approximately centrally in the wave source. This permits an inline localization which can be carried out rapidly with high precision. With the inline localization the object to be treated is imaged “from the viewpoint” of the wave source.

Preferably, a wave generator of the wave source may have a substantially ring-like shape matching the shape and/or position of the solid-state detector. The wave generator and the solid-state detector thereby efficiently exploit the available cross-sectional area of the wave source for emitting pressure waves and for receiving radiation.

Particularly advantageously, the solid-state detector can be configured to be movable inside the wave source. This permits an alignment of the solid-state detector with respect to different radiation source positions, particularly with respect to the localization of the object to be treated. The position of the wave source can here be maintained relative to the object to be treated, i.e., for instance relative to a patient. As a result, the locating operation can be carried out faster. Since the wave source can remain in its position, movements of the object to be treated, for instance a patient, which might be caused thereby, are avoided. With the aligning of the solid-state detector, its orientation relative to the radiation source can be easily adjusted for avoiding image distortions.

Preferably, it is possible to provide at least two solid-state detectors receiving radiations from different directions. A fast spatial localization is thus possible in an easy way, in the case of which the position of the wave source as such relative to the object to be treated, i.e., for instance with respect to a patient, can be maintained.

Advantageously, the treating apparatus may comprise an ultrasonic locating device integrated into the wave source. The advantages of both locating systems can thus be exploited with the treating apparatus according to the invention.

In a development of the invention, the solid-state detector may comprise at least one ultrasonic locating unit. A locating unit thereby combines both principles of localization.

Advantageously, the solid-state detector may alternately comprise radiation locating units and ultrasonic locating units. It is thereby possible to evaluate both impinging radiation and impinging ultrasound at local places of the solid-state detector.

Preferably, the solid-state detector may comprise radiation locating units and ultrasonic locating units alternating in matrix-like fashion. Both a radiation image and an ultrasonic image can thereby be created from the same position. This can possibly be done at the same time.

Advantageously, the solid-state detector may be configured in the form of a line, in the form of an area and in the form of a grid. This permits a configuration of the solid-state detector in such a manner that it is adapted to the conditions of the shockwave source and/or the requirements of pressure-wave generation and focusing. Hence, the solid-state detector can also be formed by using separate detector fragments.

### BRIEF DESCRIPTION OF THE DRAWINGS

Embodiments of the present invention are shown in the drawing and will be explained in the following.

FIG. 1 is a schematic sectional illustration of a treating apparatus of a first and a second embodiment of the invention;

FIG. 2 is a schematic sectional illustration of a treating apparatus of a third embodiment of the invention;

FIG. 3 is a schematic sectional illustration of a treating apparatus of a fourth embodiment of the invention;

FIG. 4 is a schematic sectional illustration of a treating apparatus of a fifth embodiment of the invention;

FIG. 5 is a schematic sectional illustration of a treating apparatus of a sixth embodiment of the invention;

FIG. 6 is a perspective illustration of the treating apparatus of the sixth embodiment, in a concrete configuration;

FIG. 7 is a top view on a treating apparatus of a seventh embodiment of the invention;

FIG. 8 is a schematic sectional illustration of a treating apparatus according to an eighth and ninth embodiment of the present invention; and

FIGS. 9 to 12 are illustrations of embodiments of solid-state detectors of treating apparatuses of the invention, shown in portions.

### DETAILED DESCRIPTION OF THE EXEMPLARY EMBODIMENTS

The present invention relates to a treating apparatus which can be used with a radiation source and which can particularly be employed in lithotripsy, a shockwave source being provided which generates shockwaves and with which shockwaves can be sent to an object to be treated, and a radiation locating device being also provided which is integrated into the shockwave source. To provide a treating apparatus of this type which despite the use with a radiation source permits a high image quality and accurate focusing of the shockwaves in a way which is as easy as possible, the radiation locating device comprises a radiation receiving unit which is configured as a solid-state detector. The solid-state detector can include both radiation locating units and ultrasonic locating units.

In the following description, like reference numerals shall be used for like members in the various embodiments.

A treating apparatus of the invention comprises a wave source with which acoustic energy can be produced, for instance in the form of pressure waves or shockwaves. A wave generator of the wave source is configured accordingly. This means that a wave source generally producing an acoustic energy and/or a pressure wave source and/or a shockwave source can be used as a wave source in every treating apparatus according to the invention.

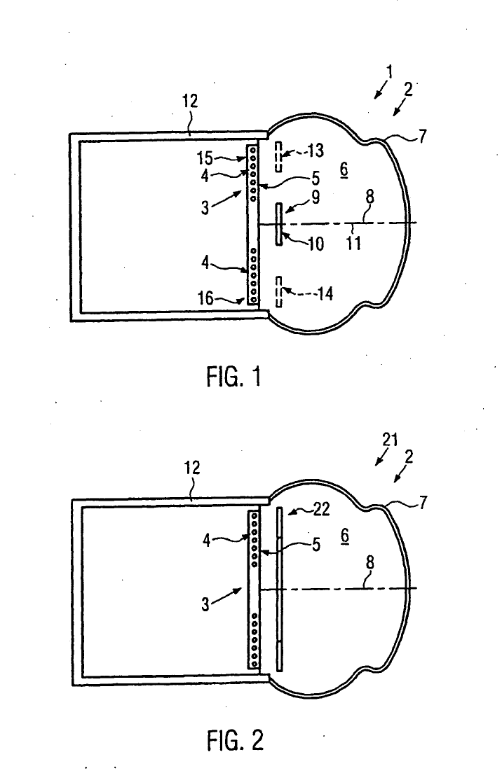

FIG. 1 is a schematic sectional illustration of a treating apparatus of a first and a second embodiment of the invention. FIG. 1 is a schematic sectional illustration showing a treating apparatus 1 of the invention which comprises a shockwave source 2 as the wave source. The shockwave source 2 includes a shockwave generator 3 as a wave generator for generating shockwaves. In this embodiment of the invention, this is carried out in accordance with the electromagnetic principle with the help of a coil 4 provided inside the shockwave generator 3. The shocks produced by the coil 4 are output with the help of a membrane 5 to a coupling medium 6 located inside a coupling bellows 7.

In all embodiments of the invention the shockwaves can also be produced in a different way, for instance with the help of electrohydraulic methods in which spark discharge takes place between electrodes, or with the help of piezoelectric methods.

The shockwaves produced by the shockwave generator are focused with the help of focusing elements (not shown) onto a focus (not shown) located outside the shockwave source 2. The focus is located in a main shockwave emission direction, which is also called shockwave path 8. The shockwave path extends approximately centrally through the membrane 5 and approximately perpendicular thereto.

It is also possible in all embodiments of the invention to use unfocused shockwaves or pressure waves, i.e. so-called ballistic shockwaves or pressure waves.

The treating apparatus 1 may be used in therapeutic applications in which pressure waves, shockwaves or generally acoustic waves are exploited, e.g. as a lithotripter. It is also possible to use the treating apparatus 1 outside the medical field in materials or components, i.e., where no patients are treated.

The treating apparatus 1 comprises a radiation receiving unit which is integrated into the shockwave source and configured as a solid-state detector 9. With the solid-state detector 9, it is possible to receive radiation emitted from a radiation source and to convert it into signals that can be further processed. For example, a radiation image can be prepared from the signals. Any radiation passing through the environment of the object to be treated and/or through the object to be treated itself can be used as radiation by the solid-state detector 9, for instance radioactive radiation and/or X-ray radiation.

In this embodiment of the invention the solid-state detector 9 can be arranged with respect to the shockwave path 8 in front of the shockwave generator 3 and approximately centrally in the shockwave source 2. It has a receiving surface 10 on which the radiation entering from the outside into the shockwave source 2, particularly through the coupling bellows 7, is observed. In this embodiment of the invention, the shockwave path 8 approximately coincides with a central axis 11 of the centrally positioned solid-state detector 9, said central axis extending transversely through the receiving area 10.

It is also possible to provide a plurality of solid-state detectors. FIG. 1 shows two solid-state detectors 13, 14 arranged in the area of external sections 15, 16 of the shockwave generator 3, in accordance with a second embodiment of the invention. The solid-state detectors 13, 14 may also be arranged between the shockwave generator 3 and a housing 12 of the shockwave source.

FIG. 2 is a schematic sectional illustration of a treating apparatus of a third embodiment of the invention. FIG. 2 shows a treating apparatus 21, in which a ring-like or a frame-like solid-state detector 22 is arranged in front of the shockwave generator 3.

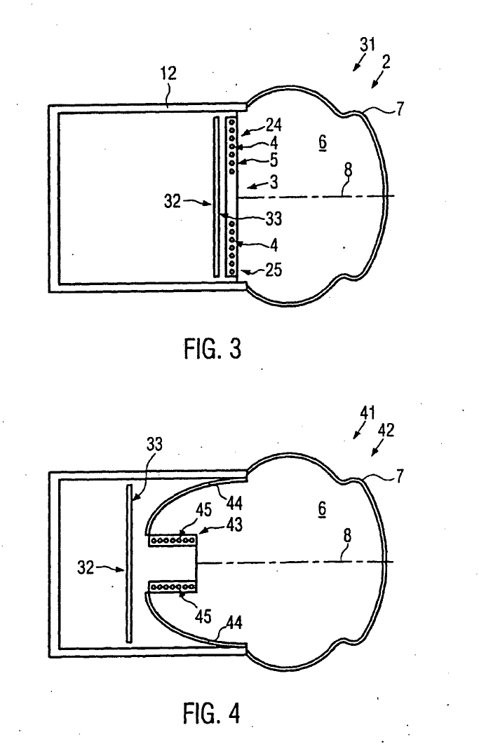

FIG. 3 is a schematic sectional illustration showing a treating apparatus 31 of the invention according to a fourth embodiment of the invention. In contrast to the first embodiment, a solid-state detector 32 is provided which is arranged relative to the shockwave path 8 behind the shockwave generator 3. The size of a receiving area 33 of the solid-state detector 32 corresponds approximately to the size of the shockwave generator 3. The membrane 5 of the shockwave generator 3 is made radiolucent, which means it is almost entirely transparent to radiation

The radiation image created with the solid-state detector 32 also covers the coil 4 of the shockwave generator 3. This, however, has no influence on image definition. Moreover, the object to be treated can still be localized without any problems.

FIG. 4 is a schematic sectional illustration showing a treating apparatus 41 of a fifth embodiment of the invention. In contrast to the fourth embodiment, a shockwave source 42 is provided with an approximately cylindrical shockwave generator 43 and a reflector 44. The shockwave generator 43 emits the shockwaves produced with the help of its coil 45 substantially in a direction transverse to the shockwave path 8, whereas in the shockwave generator 3 of the first through third embodiment this is carried out substantially in parallel with the shockwave path 8.

The shockwave generator may also be configured in accordance with the electrohydraulic principle, i.e., it may comprise an electrode array for producing spark discharges. The shockwaves are focused with the help of the reflector 44 onto the focus (not shown) located outside the shockwave source 42. The reflector 44 is made radiolucent. That is why the solid-state detector 32 can detect radiation impinging in the area of the reflector 44. When the reflector is made radiopaque, a solid-state detector may be arranged in front of or inside the reflector.

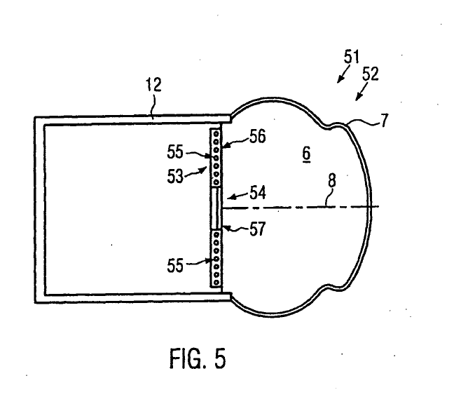

FIG. 5 is a schematic sectional illustration showing a treating apparatus 51 according to a sixth embodiment of the present invention. In contrast to the treating apparatus of the first to fourth embodiment, it comprises a shockwave generator 53 which has a solid-state detector 54 integrated thereinto. Apart from this, the shockwave source 53 is constructed like the shockwave source 2 used in the first to third embodiment. By analogy, the shockwave generator 53 comprises a coil 55 and a membrane 56. A receiving area 57 of the solid-state detector 54 is provided in a direction approximately transverse to the shockwave path 8.

The shockwave generator 53 has a substantially ring-like or frame-like shape which matches the position and shape of the solid-state detector 54. The propagation of the shockwaves emitted by the shockwave generator remains substantially unaffected by the solid-state detector in this instance. The radiation reception of the solid-state detector also remains substantially unaffected by the shockwave generator.

The receiving area 57 and the membrane 56 can be arranged to be substantially in alignment with each other.

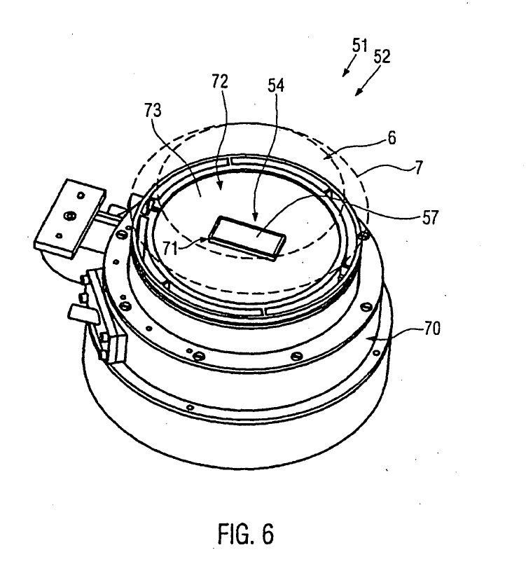

FIG. 6 is a perspective illustration of the treating apparatus of the sixth embodiment, in a concrete configuration. FIG. 6 shows the treating apparatus 51 in a concrete embodiment which comprises a shockwave source 52 as the wave source. The receiving area 57 of the solid-state detector 54 has an approximately rectangular shape. A recess 71 of an approximately rectangular shape is provided to match the rectangular shape of the solid-state detector 54. The recess 71 is provided in an acoustic lens 72 having a surface 73, which in shockwave emission direction is arranged after the shockwave generator. The solid-state detector 64 is arranged in this recess 71 and somewhat protrudes from the surface 73. The recess 71 can extend up into the shockwave generator.

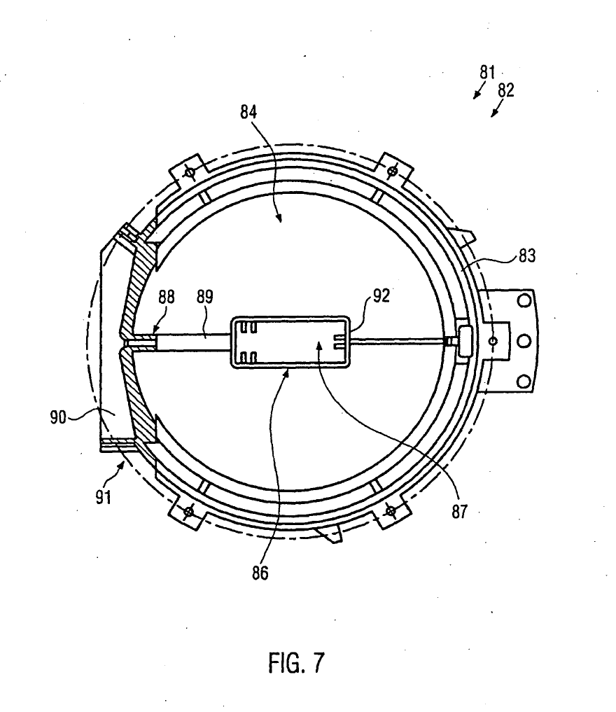

FIG. 7 shows part of a treating apparatus 81 of a seventh embodiment of the invention from above; the construction thereof is in principle identical with that of the sixth embodiment. A recess 86 in which a mount 92 of an approximately rectangular shape is received in form-fit fashion is formed in a shockwave generator 84 of the shockwave source 82. A solid-state detector 87 is integrated into the mount 92 in form-fit fashion.

The shockwave generator 84 comprises a groove 88 which accommodates a cable 89 extending to the solid-state detector 87. The cable 89 connects the solid-state detector 97 to a connector 90 which is provided on an exterior portion 91 of a housing 83 of the treating apparatus 81.

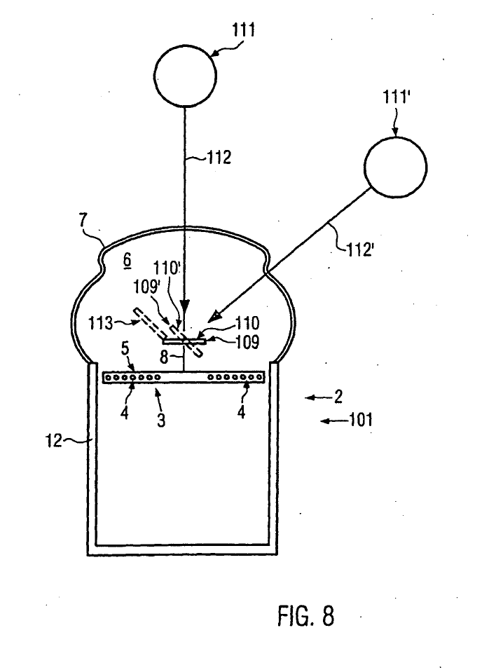

FIG. 8 is a schematic sectional illustration showing a treating apparatus 101 of an eighth embodiment of the present invention. This treating apparatus is a development of the treating apparatus 1 of the first embodiment as shown in FIG. 1. In contrast to the first embodiment, the solid-state detector is movably provided in the shockwave source 2. It can be aligned relative to different positions of radiation sources. Moreover, it can be readjusted with respect to a radiation source to avoid distortions upon receipt of radiation. FIG. 8 shows the solid-state detector with reference numeral 109 in a first position, as corresponds approximately to the position of the solid-state detector 9 in FIG. 1. In the first position the solid-state detector 109 is aligned with a radiation source 111 at a first position, a central beam 112 of the radiation source 111 extending approximately along the shockwave path 8 and impinging approximately in vertical direction on a receiving area 100 of the solid-state detector 109. Reference numeral 109′ shows the solid-state detector in broken lines in a second position in which it is aligned relative to a radiation source 111′ in a second position. The central beam 112′ of the radiation source 111′ of the second position impinges approximately in vertical direction on the receiving area 110′ of the solid-state detector 109′.

It is also possible to provide two solid-state detectors, of which each receives radiation from another (main) direction. According to a ninth embodiment of the invention, a first solid-state detector may be provided in the first position of the solid-state detector 109 of the eight embodiment; positioned at an angle and offset thereto is a second solid-state detector 113, which is shown in FIG. 8 in a dash-dotted line.

The radiation source may be provided as a radiation source which is movable between the two positions 111 and 111′ shown in FIG. 8.

Alternatively, on each of the positions 111 and 111′ shown in FIG. 8, an own radiation source may be provided. A mechanism for displacement between the two positions 111 and 111′ is here not needed.

Motion coupling is also possible between a radiation source and its associated solid-state detector for keeping the two members aligned relative to each other. However, it is also possible to provide radiation source and solid-state detector in a decoupled condition. A three-dimensional localization is here possible after all.

The radiation source and/or the shockwave source may comprise a collimator which is used in an adjusted condition in which substantially only the area of the solid-state detector is irradiated. Preferably, the collimator is adjusted to take into account the angular position of the assigned solid-state detector(s).

The radiation sources may be point, line and/or plane radiators. The latter are used, for example, in nuclear medicine.

The solid-state detectors may be angular and/or movable in all embodiments. Especially with an integrated, movable or angular configuration of the solid-state detector, the solid-state detector and/or the shockwave generator may be configured to be asymmetrical and/or may be asymmetrically arranged relative to one another.

In each embodiment of the inventive treating apparatus, the solid-state detector may be a flat panel detector.

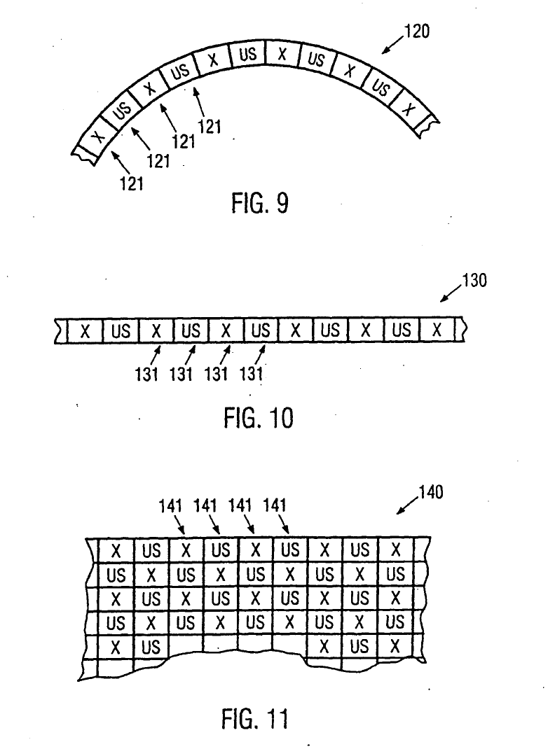

FIGS. 9 to 12 schematically show various embodiments of solid-state detectors which can be used in the treating apparatuses according to the invention.

FIG. 9 is a schematic illustration 120 of an embodiment of a solid-state detector of a treating apparatus of the invention, shown in portions. The portion of the solid-state detector shown in FIG. 9 comprises a plurality of locating units 121.

FIG. 10 is an illustration 130 of an embodiment of a solid-state detector of a treating apparatus of the invention, shown in portions. The portion of the solid-state detector shown in FIG. 10 comprises a plurality of locating units 131.

FIG. 11 is an illustration 140 of an embodiment of a solid-state detector of a treating apparatus of the invention, shown in portions. The portion of the solid-state detector shown in FIG. 11 comprises a plurality of locating units 141.

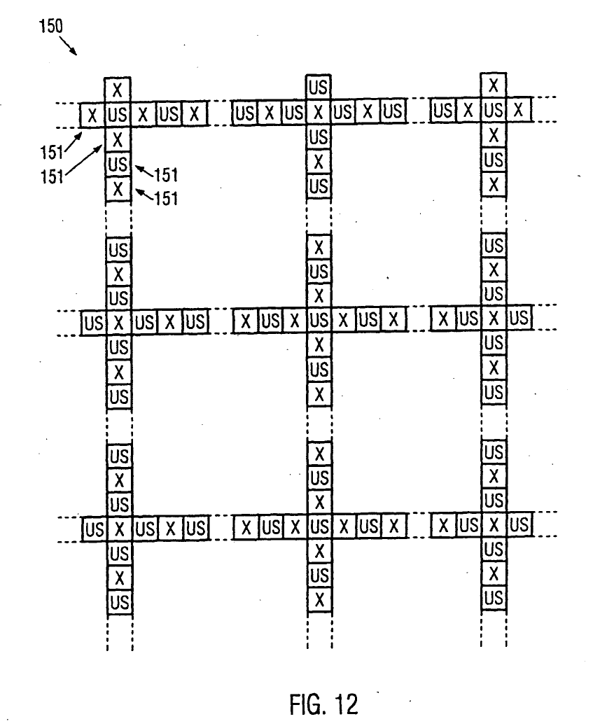

FIG. 12 is an illustration 150 of an embodiment of a solid-state detector of a treating apparatus of the invention, shown in portions. The portion of the solid-state detector shown in FIG. 12 comprises a plurality of locating units 151.

The solid-state detectors may be line-shaped, e.g. as shown in FIGS. 9 and 10. The line-like shape may be straight, as shown in FIG. 10, or curved, e.g. in the form of a circular arc, as shown in FIG. 9. As shown in FIG. 11, the solid-state detector may be configured in the form of an area. FIG. 12 illustrates a grid-like embodiment of a solid-state detector.

The solid-state detectors comprise a plurality of locating units 121, 131, 141, 151, all of which may be radiation locating units, e.g. X-ray locating units. However, radiation locating units and ultrasonic locating units may be provided that alternate at least in proportion, as illustrated in FIGS. 9 to 12, in which an “X” indicates radiation locating units and a “US” indicates ultrasonic locating units. The radiation locating units and the ultrasonic locating units may alternate one after the other, but it is also possible that a plurality of successive radiation locating units alternate with a plurality of successive ultrasonic locating units. As for solid-state detectors in the form of an area, a structure may be provided in which radiation locating units “X” alternate in the manner of a matrix with ultrasonic locating units “US,” as shown in FIG. 11.

By alternating the arrangement of radiation locating units and ultrasonic locating units, both radiation and ultrasound can be received virtually at the same place, for the locating units can be made very small. A solid-state detector comprising radiation locating units and ultrasonic locating units is a hybrid solid-state detector.

A locating mark which is also imaged on the radiation and/or ultrasound locating image may be provided on the surface of a solid-state detector according to the invention. The locating mark may have, for example, a ring-like shape and/or a crosshair shape.

In addition to a solid-state detector, a separate ultrasonic locating device may be provided in a treating apparatus according to the invention.

A control device may be provided which permits the generation of shockwaves in dependence upon the position of the shockwave source relative to the object to be treated. The control device will only permit the emission of shockwaves upon an adequately exact alignment of the shockwave source with the object to be treated. It is only in cases where the focus and the object to be treated, for instance a gallstone, substantially coincide that the control device permits the generation of shockwaves. Thus, surrounding body parts are not unnecessarily impaired by an inaccurate or incorrect alignment of the shockwave source.

The shockwave source and/or an object support, for instance a patient supporting table, may be provided in an automatically displaceable manner. The position of the shockwave source relative to the object to be treated is adjusted with the help of the control device. This may be an adjustment carried out for the first time. However, a correction of the position is also possible in this way. For instance, positional deviations resulting from a patient's movements can be corrected. Positional deviations resulting from a patient's respiration can also be compensated.

1. A treating apparatus for use with a radiation source and which can particularly be employed in lithotripsy, comprising:
a wave source which generates a plurality of pressure waves, the plurality of pressure waves sendable to an object to be treated, and
a radiation locating device which is integrated into the wave source, the radiation locating device comprising a radiation receiving unit which is configured as a solid-state detector.

2. The treating apparatus according to claim 1, wherein the solid-state detector is arranged relative to a main pressure-wave emission direction of the wave source and behind a wave generator of the wave source.

3. The treating apparatus according to claim 1, wherein the solid-state detector is arranged relative to the main pressure-wave emission direction of the wave source in front of the wave generator of the wave source.

4. The treating apparatus according to claim 1, wherein the solid-state detector is integrated into the wave generator of the wave source.

5. The treating apparatus according to claim 1, wherein the solid-state detector is arranged approximately centrally in the wave source.

6. The treating apparatus according to claim 1, wherein the wave generator of the wave source has a substantially ring-like shape matching at least one of a shape or a position of the solid-state detector.

7. The treating apparatus according to claim 1, wherein the solid-state detector is movable in the wave source.

8. The treating apparatus according to claim 1, wherein at least two solid-state detectors are provided for receiving a plurality of radiations from different directions.

9. The treating apparatus according to claim 1, wherein the treating apparatus further comprises an ultrasonic locating device, the ultrasonic locating device being integrated into the wave source.

10. The treating apparatus according to claim 1, wherein the solid-state detector comprises at least one ultrasonic locating unit.

11. The treating apparatus according to claim 1, wherein the solid-state detector comprises at least one radiation locating unit and at least one ultrasonic locating unit arranged in an alternating fashion.

12. The treating apparatus according to claim 1, wherein the solid-state detector comprises at least one radiation locating unit and at least one ultrasonic locating unit alternating in matrix-like fashion.

13. The treating apparatus according to claim 1, wherein the solid-state detector is configured in the form of at least one of:
a line, an area, and a grid.
